(19) United States
(12) Patent Application Publication
(10) Pub. No.: US 2020/0082938 A1
(43) Pub. Date: Mar. 12, 2020

(54) IN VIVO DEVICE SENSING SYSTEM

(71) Applicant: DONG-RU HO, CHIAYI COUNTY (TW)

(72) Inventors: DONG-RU HO, CHIAYI COUNTY (TW); CHIEN-HUNG LIAO, CHIAYI COUNTY (TW)

(21) Appl. No.: 16/613,809

(22) PCT Filed: May 22, 2018
(86) PCT No.: PCT/CN2018/000185

§ 371 Date: Nov. 15, 2019

Related U.S. Application Data
(60) Provisional application No. 62/509,980, filed on May 23, 2017.

(51) Int. Cl. G16H 40/67; A61B 5/00; A61B 5/145; A61B 5/1477; A61B 5/07; H01Q 1/22

(52) U.S. Cl. CPC G16H 40/67; A61B 5/0024; A61B 5/14542; H01Q 1/22; A61B 5/07; A61B 5/6804; A61B 5/1477

(57) ABSTRACT

An in vivo device sensing system telecommunicatively coupled to an in vivo device and a remote device and applied in the medical industry to obtain in vivo physiological information includes a main body, a computing module and at least one antenna module. The computing module is telecommunicatively coupled to the remote device. The antenna module is installed at the main body and telecommunicatively coupled to the in vivo device and the computing module and has plural antenna units. The antenna units receives a coordinate signal transmitted by any one of the antenna units and provided for the computing module to compute the coordinate signals and generate coordinate correction information.

### BACKGROUND

### Technical Field

The present disclosure generally relates to the field of communication and sensing. More particularly, the present disclosure relates to an in vivo device sensing system applied in the medical industry for sensing an in vivo device.

### Description of Related Art

As science and technology advance, the medical industry is developed rapidly, and scientists continue to develop a number of medical treatment devices such as an in vivo organ electrical stimulation device or an artificial organ or in vivo sensor that can be operated on human organs and tissues in a user's body in order to assist medical professionals to perform a diagnosis or treatment operation.

In general, a conventional in vivo detector has an emission source of electromagnetic signals, and the electromagnetic signals are provided for transmitting physiological information of the user's tissues or organs. When the electromagnetic signals are transmitted, the signal intensity will be decreased with an increased distance, and its attenuation rate is directly proportional to the mean square root of the distance. In addition, the distance from the sensor for transmitting and receiving the source signal to the signal source is very large, so that the intensity of the signal received by the sensor is very small. Further, a different reflective index and a different adsorption rate exist between the in vivo tissues or between the in vivo tissue and air, and thus the intensity of the signal finally received by the sensor is even smaller.

To increase the intensity of the signal received by the sensor, some scientists proposed an improved method by increasing the emitted energy of an emission source. Assumed that the sensor can receive the electromagnetic signal at a position 3 cm from the emission source, it will be necessary to increase the energy of the electromagnetic signal by 10,000 times if we want to receive the electromagnetic signal of the same intensity at a position 3 meters from the emission source. However, a vast majority of the energy of the electromagnetic signal will be attenuated gradually during the transmission process, and heat will be generated at a position near the emission source, and thus having a risk of jeopardizing the in vivo tissues or organs.

In another improved method, a wireless charging technology is adopted to learn about the position of the emission source and to supply electric energy to the emission source via wireless transmission. Even though we know the position of the emission source, the charging operation still cannot be carried out perfectly since the energy transmitted by a conventional wireless power supply is usually reflected or absorbed by human tissues. Alternatively, antenna or coil technologies are applied in an in vitro sensor to receive the electromagnetic signal of the in vivo emission source, but the aforementioned antenna sensor still has to face many challenges including the setup and the structural type of the antenna and the configuration of the antenna array such as a cross, circular, or hexagonal antenna configuration, which will affect the performance of transmitting and receiving the electromagnetic signal or the structure and appearance of the sensor. Wherein, the application of the antenna sensor has to take the positioning of the in vivo emission source into consideration.

In view of the aforementioned drawbacks of the prior art, the team of the present disclosure based on years of experience in the related industry to conduct extensive research and experiment, and finally developed an in vivo device sensing system in accordance with the present disclosure to overcome the drawbacks of the prior art.

### SUMMARY

Therefore, it is a primary objective of the present disclosure to provide an in vivo device sensing system to learn about the position of an in vivo device having an emission source and its detected electromagnetic signal, which are provided to medical professionals for diagnosis or treatment.

To achieve the aforementioned and other objectives, the present disclosure provides an in vivo device sensing system telecommunicatively coupled to an in vivo device and a remote device and applied to the medical industry to obtain in vivo physiological information, and the in vivo device sensing system comprises a main body, a computing module and at least one antenna module. The main body is provided for a user to wear. The computing module is telecommunicatively coupled to the remote device. The antenna module is installed at the main body and telecommunicatively coupled to the in vivo device and the computing module, and the antenna module has a plurality of antenna units. Wherein, the antenna units are provided for receiving a coordinate signal emitted by any one of the antenna units and received by the computing module to compute the coordinate signals and generate coordinate correction information. Wherein, any one of the antenna units receives a source signal emitted by the in vivo device and provided for the computing module to receive the source signals, and the computing module operates the source signals and the coordinate correction information to generate sensing information to be received by the remote device to display the sensing information and indicate the position and moving speed of the in vivo device in a user's body to assist medical professionals to perform a diagnosis or treatment operation, so as to improve the performance of the diagnosis or treatment operation.

In addition, the in vivo device sensing system further comprises a control module installed at the main body and telecommunicatively coupled to the computing module and the antenna module for selecting and starting at least one the antenna unit while turning off the other remaining antenna units. The control module controls the ON/OFF of the antenna units, so that the power consumption of the antenna units can be reduced, and the use of the computing module can be minimized.

Further, the control module turns on and off the antenna units with a switching time smaller than the moving time of unit distance of the in vivo device. Therefore, the moving speed of the in vivo device can be obtained accurately while saving electric energy.

In addition, the computing module has operational parameters including signal intensity, signal vector and the coordinate correction information of the source signals and the switching time of the control module to improve the accuracy of the sensing information.

In addition, the sensing information includes in vivo pressure value, pH value, temperature, drug concentration, hydrogen concentration, oxygen concentration and carbon dioxide concentration to facilitate medical professionals to perform diagnosis or treatment.

Preferably, the sensing information has image information provided for the remote device to compute and form in vivo spatial structure information to improve the performance of the diagnosis or treatment operation.

Further, each of the antenna units is substantially a square structure, a circular structure, or a hexagonal structure, and the antenna units are situated in a stacked state or an adjacent interval state to facilitate the production of the antenna units and the installation of the antenna units in the main body.

In addition, the antenna module comes with a plural quantity, and one of the antenna modules is provided for transmitting electric energy which is used for the charging operation of the in vivo device.

Preferably, the main body is a corset belt structure provided for wearing around a user's abdomen.

In summation of the description above, the in vivo device sensing system of the present disclosure is applied to the medical industry to obtain in vivo physiological information, and the antenna units and the computing module are used to learn about the position and speed of the in vivo device in a user's body accurately, so that the in vivo device sensing system can assist medical professionals in a diagnosis or treatment operation.

### BRIEF DESCRIPTION OF THE DRAWINGS

The accompanying drawings are included to provide a further understanding of the disclosure, and are incorporated in and constitute a part of this specification. The drawings illustrate embodiments of the disclosure, and together with the description, serve to explain the principles of the disclosure.

FIG. 1 is a block schematic view of a preferred embodiment of the present invention.

FIG. 2 is a flow chart of a preferred embodiment of the present invention.

FIG. 3 is an another flow chart of a preferred embodiment of the present invention.

FIG. 4 is a block schematic view of the antenna module of the present invention.

FIG. 5 is a schematic view of the present invention.

### DESCRIPTION OF THE EMBODIMENTS

Reference will now be made in detail to the present embodiments of the disclosure, examples of which are illustrated in the accompanying drawings. Wherever possible, the same reference numbers are used in the drawings and the description to refer to the same or like parts.

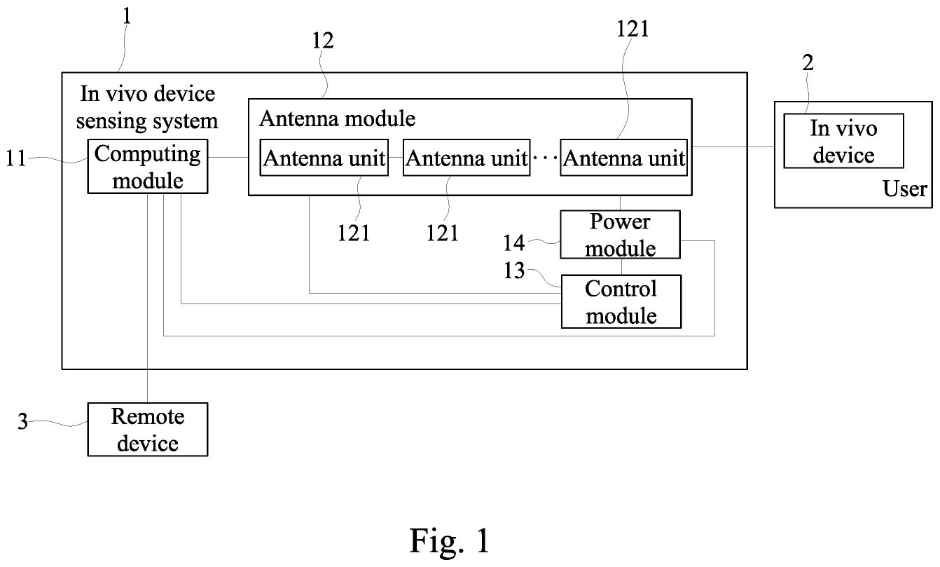

With reference to FIG. 1 for a schematic block diagram of an in vivo device sensing system 1 in accordance with a preferred embodiment of the present disclosure, the in vivo device sensing system 1 is telecommunicatively coupled to an in vivo device 2 and a remote device 3, and applied in the medical industry to obtain in vivo physiological information. The in vivo device 2 can be presented in the form of capsule having a axial radius of 1 mm-30 mm and length of 1 mm-50 mm. The in vivo device sensing system 1 comprises a main body 10, a computing module 11 and at least one antenna module 12. Wherein, the main body 10 is provided for a user to wear, and the computing module 11 is telecommunicatively coupled to the remote device 3. The antenna module 12 is installed at the main body 10 and telecommunicatively coupled to the in vivo device 2 and the computing module 11, and the antenna module 12 has a plurality of antenna units 121. In this embodiment, the computing module 11 is installed at the main body 10, and the remote device 3 comprises an arithmetic processor (not labeled in the figure) and a display device (not labeled in the figure), and the main body 10 further comprises a power module 14 electrically coupled to the computing module 11 and the antenna module 12 for supplying electric power to the in vivo device sensing system 1. Wherein, the power module 14 further comprises a sensing element (not labeled in the figure) for sensing whether or not the in vivo device 2 is situated at the neighborhood of the sensing element. In this embodiment, the sensing element adopts a method of sensing heat difference to detect whether or not there is an in vivo device 2 in the neighborhood, so as to start the power module 14. In another embodiment, the sensing element senses a pressure difference, a metal, or an electromagnetic field of the in vivo device 2 around its neighborhood, so as to further drive the ON/OFF of the power module 14.

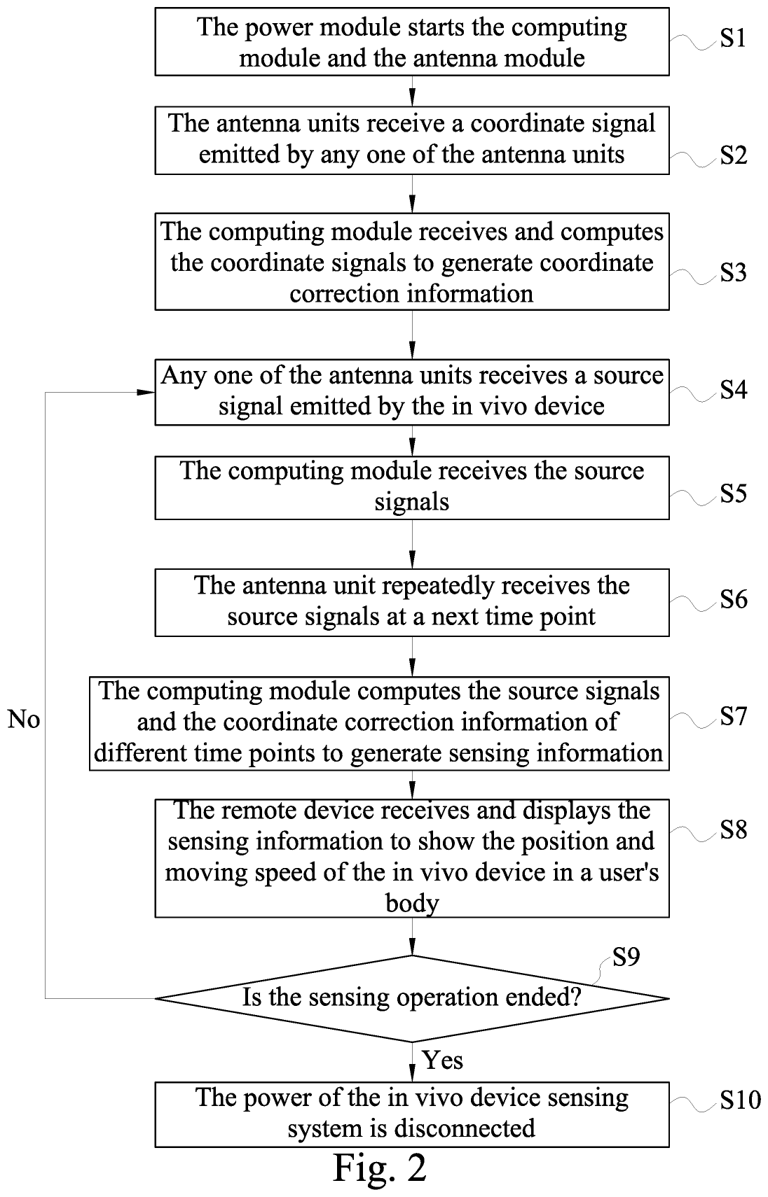

With reference to FIG. 2 for a system flow chart of a preferred embodiment of the present disclosure, when a user wears the main body 10 on the user's body and the in vivo device 2 is near the user, the power module 14 is driven to start the computing module 11 and the antenna module 12 (Step S1). Now, the in vivo device 2 has not been installed in the user's body yet, and the user has worn the main body 10 on the user's body. The antenna units 121 receive a coordinate signal emitted by any one of the antenna units 121 (Step S2). In other words, one of the antenna units 121 issues an electromagnetic signal which is the coordinate signal, while other remaining antenna units 121 receive the coordinate signals to obtain electromagnetic signals of different intensities and phase differences, so that the remaining antenna units 121 can know its relative position with respect to one of the antenna units 121. The other antenna unit 121 emits another coordinate signal to be received by the remaining antenna units 121, and this operation is repeated until each of the antenna units 121 has completed transmitting the coordinate signal. The antenna units 121 receives a plurality of different coordinate signals and provides these coordinate signals to the computing module 11, so that the computing module 11 receives and computes the coordinate signals to generate coordinate correction information (Step S3). The antenna units 121 can know their relative position to create a user's three-dimensional coordinate system.

In this embodiment, the in vivo device sensing system 1 goes through the quality control inspection before shipping. Wherein, the computing module 11 has stored the information about the relative positions between the antenna modules 12 before shipping. Further, the computing module 11 produces the coordinate correction information and knows the actual relative positions between the antenna units 121, so as to create the user's three-dimensional coordinate system during the sensing operation period. Wherein, the computing module 11 uses a look-up table and the relative position information for shipping as reference and also uses the coordinate correction information for computation, and the computing method is a linear interpolation or extrapolation or an exponential interpolation or extrapolation capable of obtaining an accurate relative positions between the antenna units 121 in the practical application. In another embodiment, the coordinate signal is an electromagnetic signal with a specific waveform such as a dumbbell-shaped electromagnetic wave, and the antenna theory is introduced to the computation of the positions of the antenna units 121 in practical applications. The computing module 11 computes the coordinate correction information to obtain the relative positions between the antenna units 121 in practical applications, so as to avoid any deviation of the position of the in vivo device 2 computed by the computing module 11 due to different body types of the users (which cause different positions of the antenna units 121 during use and result in a deviation of the computed position of the in vivo device 2).

When the in vivo device 2 is installed into a user's body by a method such as swallowing the in vivo device 2 from the mouth into the body of the user, and the in vivo device 2 moves from the esophagus to the gastrointestinal system of the user, and then discharges from the digestive system to the outside. Wherein, any one of the antenna units 121 receives a source signal emitted by the in vivo device 2 (Step S4). In other words, the antenna units 121 receive an electromagnetic signal emitted by the in vivo device 2, so that the computing module 11 receives the source signals (Step S5). In addition, the antenna units 121 repeatedly receive the source signals at a next time point (Step S6). In other words the operation as described in Step S5 is repeated to obtain a moving time difference of the in vivo device 2. The computing module 11 computes the source signals and the coordinate correction information of a different time point to generate sensing information (Step S7). In other words, the computing module 11 can receive the source signals through the antenna units 121 to obtain the relative position between the in vivo device 2 and the antenna units 121. In addition, the coordinate correction information is used to further correct further the relative position between the in vivo device 2 and the antenna units 121 in the three-dimensional coordinate system during the sensing operation period. In other words, the position of the in vivo device 2 inside the user's body can be located.

Preferably, the computing module 11 uses the aforementioned moving time difference and position of the in vivo device 2 to obtain the moving speed of the in vivo device 2 inside the user's body. Therefore, the remote device 3 receives the sensing information to display the position and moving speed of the in vivo device 2 in the user's body (Step S8). Wherein, the sensing information includes information such as the positions, the total moving time, and the moving speed of the in vivo device 2 relative to the organs or tissues inside the user's body.

If the signal intensity of the source signals is received by the computing module 11 and the error of the sensing information is too large, then the computing module 11 will generate feedback information to be sent to the antenna module 12. In this embodiment, the aforementioned signal intensity refers to the value of the source signals processed by wavelet transform to remove background noises, and the value of index size of the signal is much smaller than the value of index size of the source signals. The antenna module 12 receives the feedback information and further emits a command signal to be received by the in vivo device 2, so that the in vivo device 2 increases the signal intensity of the source signal. As a result, the intensity of the source signals that follow will be increased to facilitate the follow-up sensing operation. While computing the source signals and the coordinate correction information, the computing module 11 will also compute the feedback information to calibrate the signal intensity of the source signal to generate the sensing information. Such arrangement can avoid the error caused by a too-low signal intensity of the source signal and can facilitate the computation of the sensing information to obtain accurate in vivo physiological information.

Further, an operation controller such as a medical professional or a user can use the remote device 3 to browse the sensing information and control the in vivo device sensing system 1 to carry out a parameter correction, turn on or off the antenna units 121, and/or perform a simulation operation of the antenna module 12 or an algorithm update of the computing module 11. In addition, the operation controller may decide whether or not to end the aforementioned sensing operation (Step S9). If the sensing operation is ended, then the remote device 3 will disconnect the electric power of the in vivo device sensing system 1 (Step S10). On the other hand, if the sensing operation has not ended, then the antenna units 121 will keep on receiving the source signal. Therefore, the information such as the position and moving speed of the in vivo device 2 can be used to obtain the physiological information of the user's body to assist medical professionals to perform a follow-up diagnosis or treatment operation and improve the performance of the diagnosis or treatment operation.

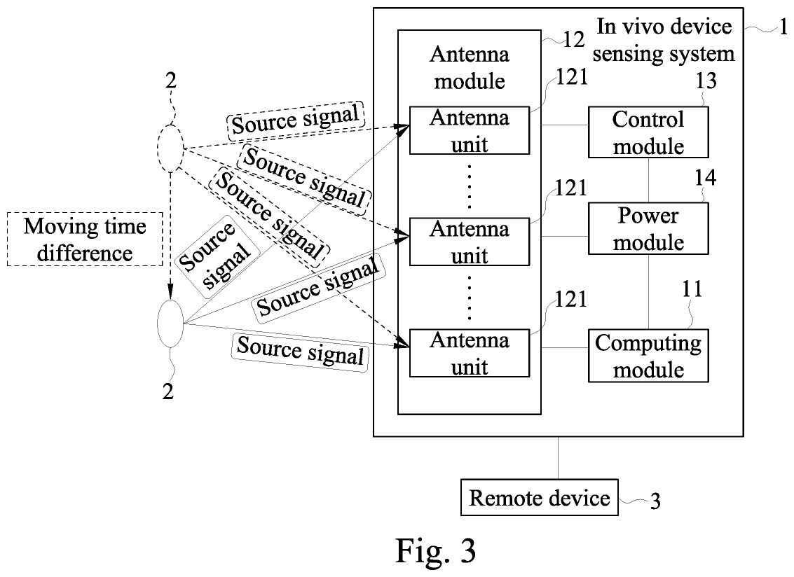

In this embodiment, the in vivo device sensing system 1 further comprises a control module 13 installed at the main body 10 and telecommunicatively coupled to the computing module 11 and the antenna module 12. With reference to FIG. 3 for the flow chart of the sensing operation in accordance with this embodiment of the present disclosure, this flow charts shows the operation of the antenna units 121 when receiving the source signals, which is also the detailed description of Steps 5 to 6 as shown in FIG. 2. When the in vivo device 2 is situated at a position indicated by a dotted line, the source signal indicated by the dotted line is emitted. Wherein, the control module 13 is provided for controlling the ON/OFF of the antenna units 121, and the control module 13 will arbitrarily select and start at least one of the antenna units 121 while turning off the remaining antenna units 121, and sequentially turn on and off the antenna units 121 until all of the antenna units 121 have received the source signals, and then the computing module 11 keeps on receiving the source signals. At a next time point, when the in vivo device 2 is moved to a position indicated by a solid line. The distance between such position and the position indicated by the dotted line position is equivalent to the moving time difference. Similarly, the control module 13 arbitrarily selects and starts the antenna unit 121 while turning off other antenna units 121, so that the antenna units 121 can receive the source signal at this time point and this position. Wherein, the control module 13 is characterized in that the switching time of turning on and off the antenna units 121 is smaller than the moving time of unit distance of the in vivo device 2. In other words, the switching time required by the control module 13 to switch any two of the antenna units 121 is less than the moving time difference. Therefore, when the position of the in vivo device 2 is detected at each time point, the antenna units 121 may be considered as performing the sensing operation at the same time. In other words, the source signal emitted by the in vivo device 2 is received indirectly at the same time. Therefore, the control module 13 controls the ON/OFF of the antenna units 121, and such arrangement not just can measure the position and speed of the in vivo device 2 accurately, but also can control the power loss of the antenna units 121, so as to lower the using frequency and production cost of the antenna units 121, while reducing the use of the computing module 11. Further, this arrangement can decrease the energy consumption rate of the antenna units 121 and reduce the volume of the in vivo device sensing system 1.

In addition, the source signal is an electromagnetic signal, so that the computing module 11 can receive the intensity and phase of the source signal, and the relative position between the antenna units 121 can be obtained by computing the coordinate correction information, and the position vector of the source signal with respect to the antenna units 121 can be known. The operational parameters of the computing module 11 include the signal intensity and signal vector of the source signals and the switching time of the coordinate correction information of the control module 11, so that the computation can be performed quickly to generate the sensing information, and the accuracy of the sensing information can be improved.

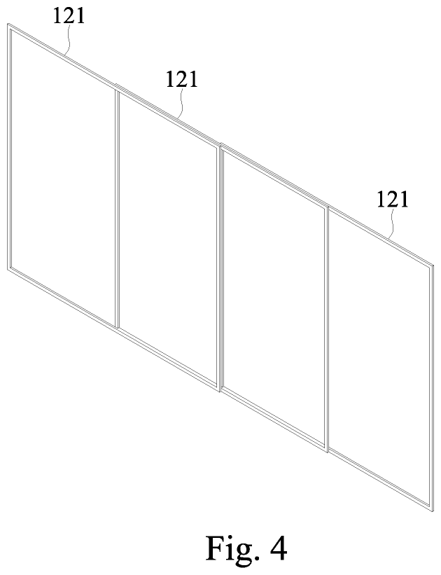

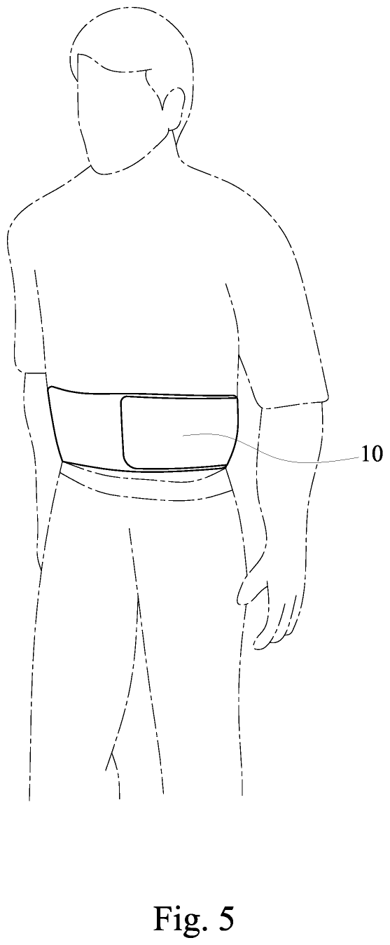

With reference to FIGS. 4 and 5 for the schematic view of the structure and the schematic view of an application of an antenna unit in accordance with a preferred embodiment of the present disclosure respectively, the main body 10 is a corset belt structure provided for a user to wear around the user's abdomen and sense the in vivo device 2 inside the user abdomen, and the antenna module 12 is disposed on the inner surface of the corset belt structure. In the figure, each of the antenna units 121 is a square structure. Since the position near the center of the square structure has weaker signal intensity, therefore the antenna units 121 are arranged in a mutually stacked state. It is noteworthy that the square structural design provides an easier way of stacking and assembling the antenna units 121 and facilitates the installation of the antenna units 121 to the main body 10. In other implementation modes, each of the antenna units 121 may be a circular structure, or a hexagonal structure, so that these antenna units 121 have different receiving powers.

Preferably, the antenna units 121 has an average edge length approximately equal to three times of the length of the in vivo device 2, and the antenna units 121 are stacked and arranged densely with each other to improve the accuracy of the source signal received by the antenna units 121. In another implementation mode, the antenna units 121 are arranged next to each other, and any two of the antenna units 121 have a constant distance apart. In another embodiment, a portion of the antenna units 121 may be in a mutually stacked state, which means there may be a small displacement at the center of the antenna units 121; as such, the antenna units 121 are not overlapping on top of each other completely. This configuration can enhance average signal intensity and coverage, and achieve better communication quality. In addition, the main body 10 is in form of a jacket, and the antenna units 121 are installed on the inner side of the main body 10 in order to be attached to the user's body and worn by the user. In other embodiments, the in vivo device 2 is installed at the user's thoracic cavity, pelvis, or arm, and the main body 10 is in form of a belt structure mounted onto the user's chest, groin and arm. Therefore, the appearance and structure of the main body 10 are designed with different forms according to the relative installation position of the in vivo device 2 and not necessarily limited to the design of the aforementioned embodiments.

In another embodiment, there are a multiple of antenna modules 12, and one of the antenna modules 12 transmits electric energy to the in vivo device 2 for performing a wireless charging operation. Preferably, the in vivo device sensing system 1 has two of the antenna modules 12, and one of the antenna modules 12 has a frequency of 2.4 GHz and is used for transmitting and receiving the coordinate signals and receiving the source signal, and the other antenna module 12 has a frequency of 433 MHz and is used for transmitting electric energy and receiving the source signal, so that the computing module 11 can receive and compute two sets of source signals which can be used as a correction reference value of its operation. In a further embodiment, the antenna module 12 used for receiving the coordinate signals and the source signal has a frequency of 433 MHz, and the other antenna module 12 used for receiving the source signal and charging has a frequency equal to 13.56 MHz, 27 MHz, or any Industrial Scientific Medical Band (ISM) frequency value, so that the antenna module is configured with more coils. Therefore, the antenna modules 12 not just can sense the in vivo device 2 only, but also can charge the vivo device 2 and prevent the failure of the in vivo device 2 caused by a low level of electric power.

In addition, the in vivo device 2 has a passive recharger device telecommunicatively coupled to the antenna modules 12. Wherein, any one of the antenna units 121 issues an initial signal to drive the passive recharger device, so as to turn on the in vivo device 2 and drive the in vivo device 2 to transmit the source signal for a sensing operation. On the other hand, electric energy is telecommunicatively transmitted to the passive recharger device for charging the antenna module 12 at the same time, maintaining the electric power of the in vivo device 2, and ensuring a normal operation of the in vivo device 2 in a user's body. During the use of the in vivo device sensing system 1, users can perform any daily activities such as sleeping or walking dogs at the same time, and the in vivo device 2 inside the users' body can perform the sensing and charging operations simultaneously. Therefore, the passive recharger device and the antenna modules 12 not just can supply power to the in vivo device 2 by the wireless charging method only, but also can avoid possible limitations of the user's daily activities caused by the position and/or the power level of the in vivo device 2 during the power supply operation. Further, the in vivo device 2 requires no battery device, and thus the total volume of the vivo device 2 can be reduced and the situation of running out of power can be avoided. Therefore, the in vivo device sensing system 1 can sense the in vivo device 2 while supplying power to the vivo device 2 at the same time.

In another implementation mode of the aforementioned embodiment, the in vivo device 2 comprises a semi-passive recharger device which is a battery part provided for actively starting the in vivo device 2 and supplying power to the antenna module 12 to carry the power supply. In addition, the battery part is detachable and further installed in an in vitro charging device such as a charging socket or a computer transmission line (USB) for the purpose of charging. Further, the battery part is installed to the in vivo device 2. Similarly, the antenna module 12 provided for charging can also be detached from the main body 10 and installed to another charging device for the purpose of charging, and then installed back to the main body 10. When the electric power level of the in vivo device 2 drops to a certain degree, such as when the power level is less than 30% of the total electric power, the battery part will be started automatically to actively supply electric power to the in vivo device 2. When the electric power of the in vivo device 2 rises to a certain degree value, such as when the power level is greater than or equal to 70% the total electric power, the antenna module 12 used for the charging purpose will be driven to start charging the in vivo device 2.

With reference to this preferred embodiment as shown in FIGS. 1 to 5, the in vivo device 2 is a device primarily used for monitoring a human digestive system, and the in vivo device 2 comprises a pressure sensor (not shown in the figure), a pH meter (not shown in the figure), a thermometer (not shown in the figure), a drug concentration detector (not shown in the figure) and a gas concentration detector (not shown in the figure) provided for measuring the pressure value, pH value, temperature value, drug concentration and gas concentration in a digestive tract respectively. Wherein, the in vivo device 2 is capable of detecting the concentration of different types of gases including common physiological gases such as hydrogen, oxygen and carbon dioxide, and the in vivo device 2 can detect the concentration of digestive drugs such as stomach medicine and gastrointestinal medicine. In another embodiment, the in vivo device 2 is capable of detecting the concentration of other different types of drugs based on a patient's tissues or organs, and the in vivo device 2 can encapsulate the aforementioned digital information into the source signal to be received by the antenna units 121. Therefore, the sensing information includes the in vivo pressure value, pH value, drug concentration and gas concentration and is provided to facilitate medical professionals to carry out follow-up diagnosis or treatment operations.

The in vivo device 2 further comprises a camera module (not shown in the figure) and a photographing lens (not shown in the figure) for photographing or video recording the in vivo tissues or organs, and their information is encapsulated into the source signal. In addition, the remote device 3 further comprises a stereoscopic reconstruction system (not shown in the figure) for computing the coordinate correction information and the sensing information to produce simulated images of the in vivo tissues or organs, wherein the simulated images are preferably created as three-dimensional structural images. The sensing information with the image information is provided for the remote device 3 to compute the image information to produce in vivo spatial structure information in order to assist medical professionals to understand the appearance of the organs or tissues in a user's body, so as to improve the performance of the diagnosis or treatment operation.

In summation of the description above, the in vivo device sensing system 1 of the present disclosure is applied in the medical industry to obtain in vivo physiological information. Wherein, the control module 13 is capable of controlling the ON/OFF of the antenna units 121, and its switching time is less than the moving time of a unit distance of the in vivo device 2. The computing module 11 and the antenna units 121 compute the coordinate correction information and the source signals to obtain information such as the position and speed of the in vivo device 2 in the user's body accurately. Therefore, the sensing information can provide the in vivo physiological information to medical professionals and users and assist the medical professionals to perform follow-up diagnosis or treatment operations.

What is claimed is:

1. An in vivo device sensing system, telecommunicatively coupled to an in vivo device and a remote device, and applied in a medical industry to obtain in vivo physiological information, comprising:
a main body, provided for a user to wear;
a computing module, telecommunicatively coupled to the remote device; and
at least one antenna module, installed at the main body and telecommunicatively coupled to the in vivo device and the computing module, and the antenna module having a plurality of antenna units;
wherein, the plurality of antenna units are provided for receiving a coordinate signal transmitted by any one of the plurality of antenna units and received by the computing module to compute the coordinate signals and generate coordinate correction information; and
any one of the plurality of antenna units receives a source signal transmitted by the in vivo device and provided for the computing module to receive the source signals, and the computing module operates the source signals and the coordinate correction information to generate a sensing information to be received by the remote device to display the sensing information, and indicate a position and a moving speed of the in vivo device in a user's body to assist medical professionals to perform a diagnosis or treatment operation.

2. The in vivo device sensing system as claimed in claim 1, further comprising a control module installed at the main body and telecommunicatively coupled to the computing module and the antenna module for selecting and starting at least one of the plurality of antenna unit while turning off the other remaining antenna units.

3. The in vivo device sensing system as claimed in claim 2, wherein the control module turns on and off the plurality of antenna units with a switching time smaller than a moving time of unit distance of the in vivo device.

4. The in vivo device sensing system as claimed in claim 3, wherein the computing module has operational parameters including signal intensity, signal vector and the coordinate correction information of the source signals and the switching time of the control module.

5. The in vivo device sensing system as claimed in claim 4, wherein the sensing information includes in vivo pressure value, pH value, temperature, drug concentration, hydrogen concentration, oxygen concentration and carbon dioxide concentration.

6. The in vivo device sensing system as claimed in claim 5, wherein the sensing information has an image information provided for the remote device to compute and form an in vivo spatial structure information.

7. The in vivo device sensing system as claimed in claim 6, wherein each of the plurality of antenna units is substantially a square structure, a circular structure, or a hexagonal structure, and the plurality of antenna units are situated in a stacked state or an adjacent interval state.

8. The in vivo device sensing system as claimed in claim 7, wherein the antenna module comes with a plural quantity, and one of the antenna modules is provided for transmitting electric energy.

9. The in vivo device sensing system as claimed in claim 1, wherein the main body is a corset belt structure provided for wearing around a user's abdomen.

10. The in vivo device sensing system as claimed in claim 2, wherein the main body is a corset belt structure provided for wearing around a user's abdomen.

11. The in vivo device sensing system as claimed in claim 3, wherein the main body is a corset belt structure provided for wearing around a user's abdomen.

12. The in vivo device sensing system as claimed in claim 4, wherein the main body is a corset belt structure provided for wearing around a user's abdomen.

13. The in vivo device sensing system as claimed in claim 5, wherein the main body is a corset belt structure provided for wearing around a user's abdomen.

14. The in vivo device sensing system as claimed in claim 6, wherein the main body is a corset belt structure provided for wearing around a user's abdomen.

15. The in vivo device sensing system as claimed in claim 7, wherein the main body is a corset belt structure provided for wearing around a user's abdomen.

16. The in vivo device sensing system as claimed in claim 8, wherein the main body is a corset belt structure provided for wearing around a user's abdomen.
